(19) United States
(12) Patent Application Publication
(10) Pub. No.: US 2018/0303545 A1
(43) Pub. Date: Oct. 25, 2018

(54) Ablation Catheter with Ultrasound Transducers for Lesion Assessment and an Ablation Method

(71) Applicant: St. Jude Medical, Cardiology Division, Inc., St. Paul, MN (US)

(72) Inventors: Fermin A. Lupotti, Lake Forest, CA (US); Stuart Rosenberg, Castaic, CA (US); John W. Sliwa, San Jose, CA (US); Riddhi Shah, San Jose, CA (US)

(21) Appl. No.: 15/768,143

(22) PCT Filed: Oct. 17, 2016
(86) PCT No.: PCT/US16/57263

§ 371 Date: Apr. 13, 2018

Related U.S. Application Data
(60) Provisional application No. 62/247,436, filed on Oct. 28, 2015.

(51) Int. Cl. A61B 18/14; A61B 8/12; A61B 8/08

(52) U.S. Cl. CPC A61B 18/1492; A61B 2018/00023; A61B 8/0833; A61B 8/12

(57) ABSTRACT

An ablation catheter (10) includes a body (12) having a distal end (16), a hollow tip (22) attached to the distal end and an ultrasound transducer assembly positioned within the hollow tip and mounted to rotate about a longitudinal axis of the catheter body. The hollow tip includes an acoustically transparent shell, which allows acoustic energy to pass to and from the ultrasound transducer assembly, and an electrically-conductive coating on its exterior surface, which allows ablating energy to be delivered to an adjacent tissue. A plurality of ribs extend inwardly from an inner surface of the shell. A system (100) incorporating the ablation catheter and methods of using the same to ablate, image and/or monitor tissue are also disclosed.

### CROSS-REFERENCE TO RELATED APPLICATIONS

This application claims the benefit of U.S. provisional application No. 62/247,436, filed 28 Oct. 2015, which is hereby incorporated by reference as though fully set forth herein.

### BACKGROUND

The instant disclosure relates generally to tissue ablation. In particular, the instant disclosure relates to systems, apparatuses, and methods for monitoring the formation of a lesion, for example in cardiac tissue, during ablation.

Catheters are used in a variety of diagnostic and therapeutic procedures, for example to diagnose and/or treat conditions such as atrial arrhythmias. For example, a catheter carrying one or more electrodes can be deployed and manipulated through a patient's vasculature and, once located at the intended site, radiofrequency (“RF”) energy can be delivered through the electrodes to ablate tissue.

In some catheters, an additional sensor, such as an ultrasound sensor, is provided in the catheter tip to provide additional information during the primary diagnosis or therapy. For example, practitioners often desire information about lesion formation, such as lesion depth, lesion transmurality, and pop potential. Thus, RF ablation catheters can include one or more sensors (e.g., ultrasound sensors; optical sensors), sometimes located within the hollow tip of the catheter, that can be used to monitor the progress of a lesion forming in the tissue being treated and/or to confirm one or more characteristics of the lesion once created.

### BRIEF SUMMARY

Disclosed herein is an ablation catheter, including: a catheter body having a distal end; a hollow tip attached to the distal end of the catheter body, the hollow tip including an acoustically transparent shell and an electrically-conductive coating on an exterior surface of the shell; and an ultrasound transducer assembly positioned within the hollow tip and mounted to rotate about a longitudinal axis of the catheter body, wherein the ultrasound transducer assembly includes: a first ultrasound transducer oriented more forward-looking than side-looking relative to the hollow tip; and a second ultrasound transducer oriented at at least a 45 degree angle relative to a longitudinal axis of the catheter body.

According to aspects of the disclosure, the shell also includes a plurality of ribs extending inwardly from an inner surface of the shell. The ribs can be spaced at constant intervals about a perimeter of the shell.

In certain embodiments, the plurality of ribs can be integrally formed with the shell. Alternatively, and in other embodiments, the ribs can be attached to the inner surface of the shell.

According to aspects of the disclosure, the plurality of ribs include metallic or metal-reinforced ribs. It is also contemplated, however, that the ribs can include ceramic or ceramic-reinforced ribs.

Dimensionally, it is desirable for the ribs to increase a local thickness of the shell by a factor of at least 3 (i.e., the thickness of the rib, as it extends into the interior of the shell, is about twice the thickness of the portion of the shell at which the rib originates). The width of each rib can be about equal to the thickness of the shell itself, exclusive of the rib.

The shell can be polymeric, such as polymethylpentene. The electrically-conductive coating can include titanium, chrome, and/or gold.

The catheter can also include a sensor for measuring a rotational attitude of the ultrasound transducer as it rotates about the longitudinal axis of the catheter body.

Also disclosed herein is a method of ablating tissue. The method includes: providing an ablation catheter, such as described above; supplying ablating energy to a tissue to be ablated through the electrically-conductive coating on the exterior surface of the shell; and monitoring the tissue to be ablated via the ultrasound transducer assembly while rotating the ultrasound transducer assembly about the longitudinal axis of the catheter body.

The step of monitoring the tissue to be ablated can include using the plurality of ribs to determine a rotational attitude of the ultrasound transducer assembly as it rotates about the longitudinal axis of the catheter body. The step of monitoring the tissue to be ablated can also include monitoring progress of a lesion forming in the tissue to be ablated; the method can also include adjusting an amount of the ablating energy supplied to the tissue to be ablated responsive to the monitored progress of the lesion forming in the tissue to be ablated (that is, the monitoring can be used as part of a feedback control loop).

It is also contemplated that the method can include imaging the tissue to be ablated via the ultrasound transducer assembly while rotating the ultrasound transducer assembly about the longitudinal axis of the catheter body.

The present disclosure also provides an ablation and lesion feedback system, including: an ablation catheter, such as described above; and a control unit. The control unit can be configured to: energize the electrically-conductive coating on the exterior surface of the shell to deliver ablating energy to a tissue to be ablated; and operate the ultrasound transducer assembly to monitor the tissue to be ablated as the ultrasound transducer assembly rotates about a longitudinal axis of the catheter body. The system can also include a radiofrequency energy source coupled to the electrically-conductive coating (e.g., to supply ablating energy thereto) and the control unit (e.g., for control purposes); a transducer pinger coupled to the ultrasound transducer assembly (e.g., to cause the ultrasound transducer assembly to emit acoustic energy) and the control unit (e.g., for control purposes); and/or an acoustic receiver coupled to the ultrasound transducer assembly (e.g., to receive acoustic echoes from the ultrasound transducer assembly) and the control unit (e.g., for control purposes).

The foregoing and other aspects, features, details, utilities, and advantages of the present invention will be apparent from reading the following description and claims, and from reviewing the accompanying drawings.

### BRIEF DESCRIPTION OF THE DRAWINGS

FIG. 1 depicts an ablation and lesion feedback system including an exemplary catheter including a sensor-bearing tip.

FIG. 2 is a partial cut-away view of a sensor bearing hollow catheter tip according to embodiments of the instant disclosure.

FIG. 3A is a transverse cross-sectional view of a first embodiment of a hollow catheter tip according to aspects of the instant disclosure.

FIG. 3B is a cross-section taken along line 3B-3B in FIG. 3A.

FIG. 4A is a transverse cross-sectional view of a second embodiment of a hollow catheter tip according to aspects of the instant disclosure.

FIG. 4B is a cross-section taken along line 4B-4B in FIG. 4A.

FIG. 5A is a transverse cross-sectional view of a third embodiment of a hollow catheter tip according to aspects of the instant disclosure.

FIG. 5B is a cross-section taken along line 5B-5B in FIG. 5A.

FIG. 6 is a partial cut-away view of an irrigated sensor bearing hollow catheter tip according to aspects disclosed herein.

### DETAILED DESCRIPTION

The present disclosure provides methods, apparatus, and systems for monitoring the formation of a lesion in tissue. For purposes of illustration, several exemplary embodiments will be described herein in detail in the context of a radiofrequency (“RF”) ablation catheter including an acoustic sensor (e.g., a pulse-echo ultrasound transducer) that can be used to monitor the progress of the thermal lesion being formed in an adjacent tissue. It should be understood, however, that the methods, apparatuses, and systems described herein can be utilized in other contexts. For example, optical sensors can be used as an alternative or in addition to the ultrasound transducers described herein.

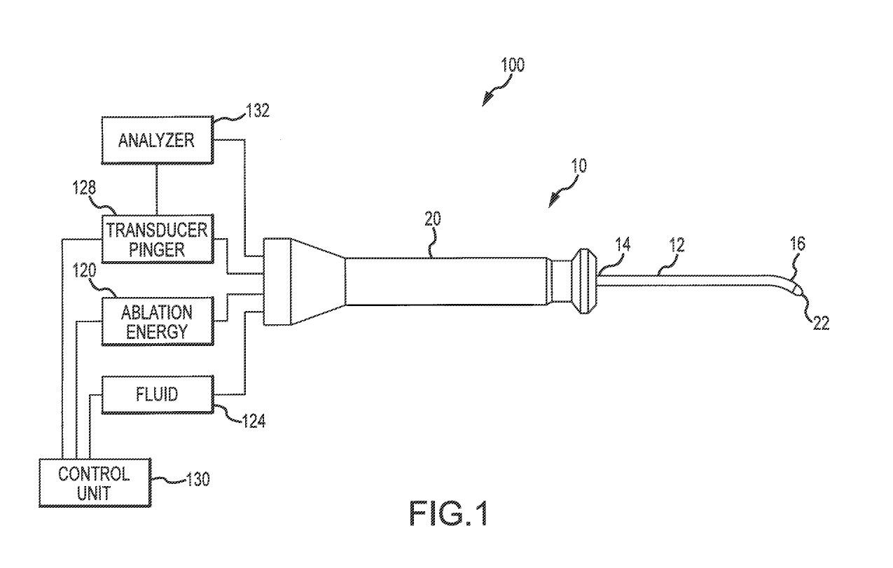

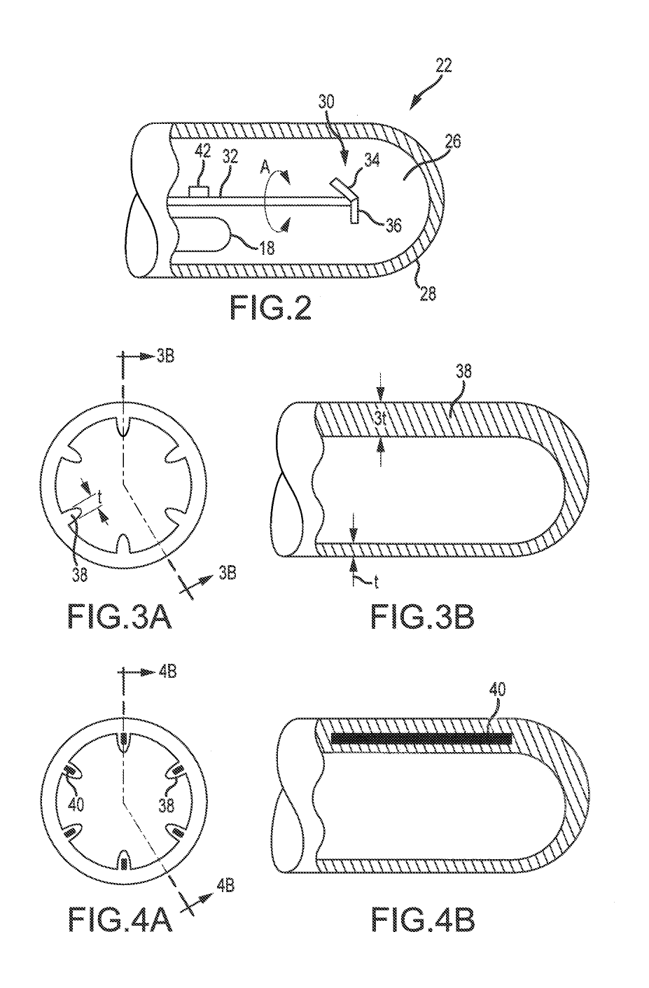

FIG. 1 is a schematic diagram of an ablation and lesion feedback system 100 including an exemplary catheter 10. As shown in FIG. 1, catheter 10 generally includes an elongated, hollow, and flexible tubular body 12 having a proximal end 14 and a distal end 16. Tubular body 12 defines a lumen 18 (not visible in FIG. 1, but visible, inter alia, in FIG. 2). Although only a single lumen 18 is depicted in certain figures for clarity of illustration; it should be understood that any number of lumens 18 can be used without departing from the scope of the instant teachings. As the person of ordinary skill in the art will appreciate, tubular body 12 can also contain electrical interconnect wires, pull-wires, and the like.

Proximal end 14 of tubular body 12 is attached to a catheter control handle 20. Catheter control handle 20 can include, for example, an actuator (not shown) coupled to suitable structure (e.g., pull wires and/or pull rings) within tubular body 12 in order to effect the deflection of distal end 16 in one or more bending planes. It can also include electrical power and/or signal connections to additional components of ablation system 100 as discussed in further detail below.

A hollow tip 22 is attached to distal end 16 of tubular body 12. Various embodiments of tip 22 will be described in further detail below with reference to FIGS. 2-6. In general, however, for purposes of the instant disclosure, tip 22 can include an RF ablation element, such as a tip electrode, and can, as such, be connected with an ablation energy source 120, such as an RF generator.

FIG. 2 is a partial cut-away view of hollow tip 22 according to certain aspects of the instant disclosure. As shown in FIG. 2, an irrigant lumen 18 of tubular body 12 is in fluid communication with the interior 26 of hollow tip 22, which is defined by a wall 28 of hollow tip 22. An irrigant (e.g., saline) or other fluid can be delivered from fluid source 124 (shown in FIG. 1), through lumen 18, and into hollow tip 22, for example for cooling purposes, for energy transmission purposes, and/or for acoustic matching purposes. An additional lumen 18, such as an irrigant return lumen, and/or irrigant egress ports in wall 28, can also be included.

Wall 28 of hollow tip 22 defines a shell, which is at least acoustically translucent and is optionally acoustically transparent. Typically, wall 28 will be non-metallic; in embodiments, wall 28 is made of a polymer, such as polymethylpentene (e.g. TPX®), which is nearly transparent to ultrasound. Wall 28 can be either machined or molded, though molding is more cost effective and facilitates the incorporation of the strengthening ribs described below. In certain embodiments of the disclosure, at its thinnest points, wall 28 can be between about 0.004 and 0.008 inches thick.

A thin electrically-conductive coating is deposited on the outer surface of wall 28, such that hollow tip 22 can be used as an ablation electrode despite being largely made of a non-electrically-conductive material (e.g., polymer). For example, a thin layer of 500 Angstroms titanium/4000 Angstroms gold or titanium or 500 Angstroms chrome/4000 Angstroms gold or platinum can be deposited on the outer surface of wall 28, such as by sputtering. Despite their higher acoustic impedance, such metallic films minimally impact the overall acoustic transparency of wall 28, insofar as they are but a tiny fraction of the thickness of wall 28 (for example, on the order of less than about 0.5% of the thickness of wall 28 at its thinnest point).

FIG. 2 also depicts an ultrasound transducer assembly 30 positioned within interior 26 of hollow tip 22. Ultrasound transducer assembly 30 is mounted to rotate about a longitudinal axis of body 12 (arrow “A”), for example by attaching ultrasound transducer assembly 30 to a drive shaft 32.

It should be understood that, as used herein, the term “rotate” is not limited to continuous, 360 degree revolutions of drive shaft 32 about the longitudinal axis of body 12. Rather, the term “rotate” is used broadly to encompass not only continuous revolutions of drive shaft 32, but also various oscillatory arcs or incremental angular steps of drive shaft 32 in any rotational direction(s).

As shown in FIG. 2, ultrasound transducer assembly 30 includes multiple ultrasound transducers 34, 36. Ultrasound transducers 34, 36 may be pulse/echo transducers and can include a piezomaterial layer, an attenuative backer, and one or more acoustic matching layers. Insofar as these features of an ultrasound transducer will be familiar to those of ordinary skill in the art, they are not further explained herein.

Ultrasound transducer 34 is oriented such that it emits and receives acoustic energy along a beam path that forms at least a 45 degree angle relative to the longitudinal axis of catheter body 12. Thus, as drive shaft 32 rotates, ultrasound transducer 34 will image in a three dimensional slice (e.g., a substantially cone-shaped slice).

Ultrasound transducer 36, on the other hand, is oriented to be more forward-looking than side-looking, and can, in embodiments, be oriented such that it emits and receives acoustic energy along a beam path that is substantially parallel to the longitudinal axis of catheter body 12. This, as drive shaft 32 rotates, ultrasound transducer 36 will image in substantially the same direction.

In certain embodiments of the disclosure, the fields of view of ultrasound transducers 34, 36 will abut or slightly overlap, in order to minimize “dead” space where tissue is not imaged. It will be appreciated that any transducer beam has a beam thickness; thus, any image slice made with the transducer has a finite slice thickness.

It should also be understood that ultrasound transducers 34, 36 can be any suitable ultrasound transducer. For example, according to aspects of the disclosure, ultrasound transducers 34, 36 can be single-element transducers. In other aspects of the disclosure, ultrasound transducers can be individually-focused (e.g., spherical concave focused transducers; flat transducers with acoustic lenses) or individually-unfocused.

Further, although only two ultrasound transducers 34, 36 are shown and described herein, it should be understood that additional ultrasound transducers can be included in ultrasound transducer assembly 30 without departing from the scope of the instant teachings. For example, in embodiments of the disclosure, a third ultrasound transducer is provided and oriented at an angle between that of ultrasound transducer 34 (e.g., about 45 degrees to the longitudinal axis) and that of ultrasound transducer 36 (e.g., parallel to the longitudinal axis).

In addition, ultrasound transducers 34, 36 require electrical connections routed back to handle 20 for interconnection to other elements of system 100. In some embodiments, rotary transformers (not shown) are mounted on drive shaft 32, such as is the case in many extant intravascular ultrasound (“IVUS”) catheters. Of course, a suitable wireless communication protocol could also be used to send and receive signals from ultrasound transducers 34, 36.

In general, however, the person of ordinary skill in the art will appreciate from the teachings herein how to select and configure ultrasound transducer assembly 30 for particular uses of catheter 10, and thus further explanation specific to ultrasound transducer assembly 30 herein will be limited to that necessary to the understanding of the present disclosure.

A transducer pinger 128 (see FIG. 1), which might have more than one transducer channel, supplies pinging energy, such as electrical voltage pulses, to transducer assembly 30 (e.g., to ultrasonic transducers 34, 36 sequentially or simultaneously). A control unit 130 (also shown in FIG. 1) is provided for controlling the ablation and the acoustic pinging during ablation. For instance, control unit 130 can be configured to carry out duty cycling or synchronization for both ablation and pinging. (The term “pinging” is used herein to mean transmitting acoustic energy and then receiving the reflected or echoed acoustic energy.) Control unit 130 can also be configured to synchronize acoustic pinging with rotation of drive shaft 32/ultrasound transducer assembly 30.

An acoustic pinger echo analyzer or acoustic receiver 132 is provided to condition and analyze the data collected by transducer assembly 30 to provide lesion feedback. Although separate lines are shown in FIG. 1 as interconnecting the various components of lesion feedback system 100, it is contemplated that the transmit pulse and the receive pulse may be carried by the same interconnection wire or cable, such as a microcoaxial cable having an inner “hot” conductor and an outer “ground” shield.

The information can be presented to a practitioner (e.g., as an image using a graphical user interface) to provide real-time assessment of the ablation target, the ablation process, and/or the ablation result. It should be understood that the lesion being monitored and/or imaged will most likely be forming/formed on the tissue-contacting side of catheter 10, and thus will be seen in the image on that side of catheter 10. The image will also aid the practitioner in verifying tissue contact before initiating ablation. Any manner of presenting the image to a user, including, without limitation, two- and three-dimensional imagery, is within the scope of this disclosure.

Acoustic receiver 132 can also be used to identify and reject acoustic signals oriented towards/emanating from the blood pool versus the tissue being ablated. For example, a portion of the cone-shaped three dimensional slice imaged by ultrasound transducer 34 will capture tissue being ablated (e.g., the tissue against which hollow tip 22 is juxtaposed), while the rest of the cone will be oriented towards the blood pool. Acoustic signals oriented towards/emanating from the tissue, which will have a higher-intensity reflection at a much shorter time period as compared to acoustic signals oriented towards/emanating from the blood pool; acoustic receiver 132 can apply signal processing algorithms that leverage this characteristic to identify, retain, process, and output only data regarding the tissue being ablated and, optionally, surrounding and/or adjacent tissues.

In addition, acoustic receiver 132 can apply signal processing algorithms to classify sub-regions within imaged zones as one or more of blood, healthy myocardium, scarred myocardium, lesioned myocardium, pericardium, coagulum, char, steam bubble, and extracardiac structures. Such classification can be presented to a user as part of an image of the tissue, for example by color-coding the image

The various information described above may additionally or alternatively be used by system 100 to issue warning/alarms to a practitioner or as input to a feedback control loop under the control of control unit 130 that controls ablation power, irrigant flow, and the like to avoid steam pops, to reach a desired lesion depth, or to achieve another diagnostic or therapeutic objective.

Thus, one aspect disclosed herein is directed to an RF ablation catheter including ultrasound transducers capable of inter alia, acoustic feedback of the lesion and/or tissue contact feedback. The catheter is also capable of delivering an RIF ablating tip to a patient's tissue to be ablated. These aspects and others are also described in U.S. provisional application No. 62/113,833 and United States patent application publication no. 2012/0265069, both of which are hereby incorporated by reference as though fully set forth herein.

To facilitate imaging using transducer assembly 30, and as discussed above, it is desirable to minimize the thickness of wall 28 to minimize acoustic attenuation therethrough. This is particularly desirable at frequencies between about 7 Mhz and 60 Mhz, which provide excellent image resolution.

In order to improve the structural integrity of hollow tip 22, FIGS. 3A and 3B illustrate an embodiment of hollow tip 22 that includes a plurality of ribs 38 extending inwardly from the inner surface of wall 28.

As shown in FIGS. 3A and 3B, ribs 38 are integrally formed as part of wall 28 (e.g., they are part of the mold used to form wall 28, of the same material as wall 28, and thus co-molded with wall 28). FIGS. 3A and 3B also show that ribs 38 effectively locally triple the thickness of wall 28 (e.g., if the minimum wall thickness is about 0.005″, then the ribs have a thickness of about 0.015″ inclusive of wall 28) at various points; as shown, there are six ribs spaced equally around wall 28, but other arrangements and numbers of ribs 38 are contemplated as within the scope of the instant disclosure.

In addition, each rib 38 can have a width that is approximately equal to or somewhat greater than the nominal thickness of wall 28 (e.g., if the minimum wall thickness is about 0.005″, then the width of rib 38 is also about 0.005″). In other embodiments, the minimum thickness of wall 28 is about 0.006″, the thickness of each rib 38 is about 0.018″ inclusive of wall 28), and the width of each rib 38 is about 0.012″.

Ribs 38 substantially increase the crush-fracture and bending-fracture strength of hollow tip 22. They also allow higher-pressure irrigation flushing of hollow tip 22. Due to their relatively narrow widths, however, ribs 38 block only a small fraction of the view of ultrasound transducer assembly 30 through wall 28, even if there are several ribs 38 attached to wall 28. Further, as discussed below, the acoustic reflections of ribs 38 can act as a form of rotary encoder for sensing the rotational attitude of drive shaft 32/ultrasound transducer assembly 30.

FIGS. 4A and 4B show an alternative embodiment of hollow tip 22 including ribs 38 that include an embedded or laminated reinforcing structure 40. Reinforcing structure 40, which may be made of metal (e.g., stainless steel; nitinol) or another suitable material, provides additional stiffening and strength to hollow tip 22. According to aspects of the disclosure, wall 28 is overmolded over reinforcing structure 40. For example, wall 28 can be overmolded over a stainless steel cage, and pieces of the cage outside of wall 28 and ribs 38 can then be trimmed away after they have served the purpose of aligning and/or affixing reinforcing structure 40 to wall 28. Metallic ribs further enhance the strength of hollow tip 22, and can also be utilized to carry ablating energy to the electrically-conductive coating, for example through vias in wall 28.

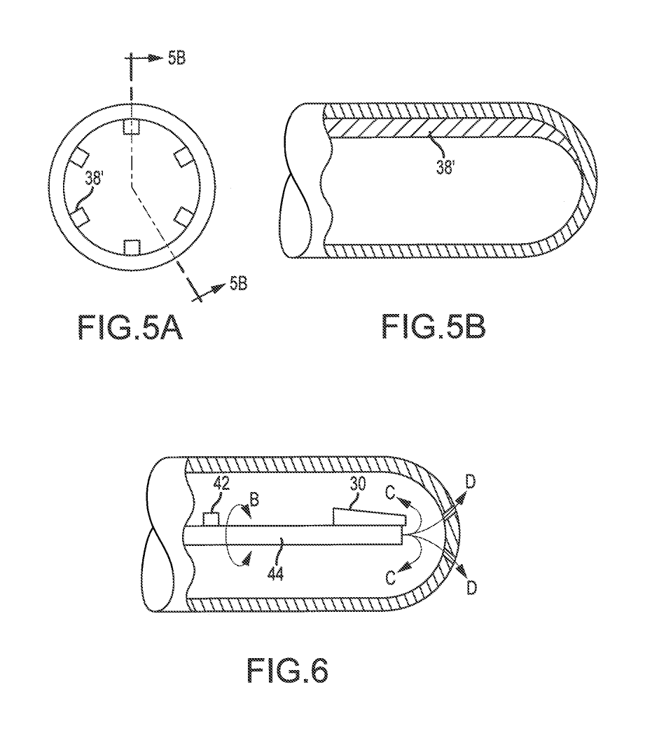

In still other embodiments, the ribs are formed separately from, and thereafter attached (e.g., laminated) to, the inner surface of wall 28. For example, FIGS. 5A and 5B illustrate a plurality of ribs 38′ that are attached to the inner surface of wall 28. Ribs 38′ may be metallic (e.g., stainless steel; nitinol), ceramic, or of another suitable material. Ribs 38′ may be attached to the inner surface of wall 28 via. adhesive (e.g., epoxy), via thermal bonding, via ultrasonic bonding, or via any other suitable method. It is also contemplated that ribs 38′ may be formed by attaching a cage (e.g., a stainless steel cage) to the inner surface of wall 28 and then trimming away undesired/unneeded portions of the cage, leaving only ribs 38′. As another advantage, metallic ribs 38′ can assist in heat removal from hollow tip 22 and the delivery of RF energy for thermal ablation of adjacent tissue, as described. above.

As discussed above, there will be rotational attitudes of ultrasound transducer assembly 30 that result in acoustic energy being emitted and/or received toward and/or through ribs 38 (or ribs 38′, in the embodiment of FIGS. 5A and 5B), The ribs, however, have different acoustic transmission properties than the remainder of wall 28. For example, an acoustic beam may scatter about a rib 38. As another example, metallic (38′) or metal-reinforced ribs (as shown in FIGS. 4A and 4B) may even be acoustically opaque. The presence of ribs can be taken into consideration when processing received acoustic echoes at analyzer 132; ribs 38 can be identified within the image as regions with minimal or no return signal from tissue or blood.

In embodiments of the disclosure, one or more rotational sensors 42 are in communication with analyzer 132. In particular, rotational sensors 42 measure the rotational attitude of ultrasound transducer assembly 30, for example by measuring the rotational attitude of drive shaft 32, and provide that information to analyzer 132, so that it can be taken into consideration when processing received acoustic echoes.

In other aspects of the disclosure, drive shaft 32 is driven by a stepper motor or servo motor, such that the various rotational orientations of ultrasound transducer assembly 30 are known (e.g., by a rotor-integrated encoder).

Alternatively, a dedicated rotary encoder, which can be mechanical, optical, magnetic, capacitive, or of any other suitable technology can be used to output the rotational attitude of ultrasound transducer assembly 30. Indeed, it is contemplated that ribs 38 themselves can function in the nature of a rotary encoder; one of the ribs 38 may have a somewhat different width than the others in order to designate a “home” rotational attitude.

In any event, by providing data regarding the rotational attitude of ultrasound transducer assembly 30, analyzer 132 can adapt the image it provides to a practitioner, for example by discarding acoustic echoes received through ribs 38 and displaying those arcs of the image as blank space.

According to aspects of the disclosure, catheter 10 is irrigated, for example via irrigant lumen 18 depicted in FIG. 2. FIG. 6 depicts another irrigated embodiment of the instant disclosure. As shown in FIG. 6, irrigation lumen 44, to which ultrasound transducer assembly 30 is mounted, is able to rotate (arrow B) about its longitudinal axis. That is, in the embodiment of FIG. 6, irrigation lumen functions in the nature of drive shaft 32 described above. Irrigant can exit the distal end of irrigation lumen 44 and be recycled (arrows C), exhausted in vivo through irrigation apertures in wall 28 (arrows D), or a combination of both.

The foregoing methods can be carried out by one or more processors incorporated into control unit 130 and/or analyzer 132. As used herein, the term “processor” refers to not only a single central processing unit (“CPU”), but also to a plurality of processing units, commonly referred to as a parallel processing environment. It should also be understood that the methods disclosed herein can be hardware and/or software implemented.

Although several embodiments of this invention have been described above with a certain degree of particularity, those skilled in the art could make numerous alterations to the disclosed embodiments without departing from the spirit or scope of this invention.

For example, although the description above relates to providing a practitioner with feedback regarding lesion formation, the lesion formation information can also be used to automatically control the ablation process (e.g., control unit 130 can be programmed to discontinue the application of ablative energy when the lesion being formed in the adjacent tissue reaches a desired depth).

As another example, where it is desirable to have feedback regarding the temperature within hollow tip 22, one or more temperature sensors, such as thermocouples or thermistors, can be placed within ribs 38 so as not to weaken wall 28.

All directional references (e.g., upper, lower, upward, downward, left, right, leftward, rightward, top, bottom, above, below, vertical, horizontal, clockwise, and counterclockwise) are only used for identification purposes to aid the reader's understanding of the present invention, and do not create limitations, particularly as to the position, orientation, or use of the invention. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other.

It is intended that all matter contained in the above description or shown in the accompanying drawings shall be interpreted as illustrative only and not limiting, Changes in detail or structure may be made without departing from the spirit of the invention as defined in the appended claims.

What is claimed is:

1. An ablation catheter, comprising:
a catheter body having a distal end;
a hollow tip attached to the distal end of the catheter body, the hollow tip comprising an acoustically transparent shell and an electrically-conductive coating on an exterior surface of the shell; and
an ultrasound transducer assembly positioned within the hollow tip and mounted to rotate about a longitudinal axis of the catheter body, wherein the ultrasound transducer assembly comprises:
a first ultrasound transducer oriented more forward-looking than side-looking relative to the hollow tip; and
a second ultrasound transducer oriented at at least a 45 degree angle relative to a longitudinal axis of the catheter body.

2. The ablation catheter according to claim 1, wherein the shell comprises a plurality of ribs extending inwardly from an inner surface of the shell.

3. The ablation catheter according to claim 2, wherein the plurality of ribs are spaced at constant intervals about a perimeter of the shell.

4. The ablation catheter according to claim 2, wherein the plurality of ribs are integrally formed with the shell.

5. The ablation catheter according to claim 2, wherein the plurality of ribs are attached to the inner surface of the shell.

6. The ablation catheter according to claim 5, wherein the plurality of ribs comprise a plurality of metallic ribs.

7. The ablation catheter according to claim 5, wherein the plurality of ribs comprise a plurality of ceramic ribs.

8. The ablation catheter according to claim 2, wherein the plurality of ribs are metal-reinforced.

9. The ablation catheter according to claim 2, wherein each rib of the plurality of ribs increases a thickness of the shell by a factor of at least 3.

10. The ablation catheter according to claim 2, wherein a width of each rib of the plurality of ribs is at least equal to a thickness of the shell.

11. The ablation catheter according to claim 1, wherein the shell comprises a polymeric shell.

12. The ablation catheter according to claim 11, wherein the polymeric shell comprises polymethylpentene.

13. The ablation catheter according to claim 1, further comprising a sensor for measuring a rotational attitude of the ultrasound transducer as it rotates about the longitudinal axis of the catheter body.

14. A method of ablating tissue, comprising:
providing an ablation catheter, the ablation catheter comprising:
a catheter body having a distal end;
a hollow tip attached to the distal end of the catheter body, the hollow tip comprising an acoustically transparent shell and an electrically-conductive coating on an exterior surface of the shell;
a plurality of ribs extending inwardly from an inner surface of the shell; and
an ultrasound transducer assembly positioned within the hollow tip and mounted to rotate about a longitudinal axis of the catheter body;
supplying ablating energy to a tissue to be ablated through the electrically-conductive coating on the exterior surface of the shell; and
monitoring the tissue to be ablated via the ultrasound transducer assembly while rotating the ultrasound transducer assembly about the longitudinal axis of the catheter body.

15. The method according to claim 14, wherein monitoring the tissue to be ablated comprises using the plurality of ribs to determine a rotational attitude of the ultrasound transducer assembly as it rotates about the longitudinal axis of the catheter body.

16. The method according to claim 14, wherein monitoring the tissue to be ablated comprises monitoring progress of a lesion forming in the tissue to be ablated, and wherein the method further comprises adjusting an amount of the ablating energy supplied to the tissue to be ablated responsive to the monitored progress of the lesion forming in the tissue to be ablated.

17. The method according to claim 14, further comprising imaging the tissue to be ablated via the ultrasound transducer assembly while rotating the ultrasound transducer assembly about the longitudinal axis of the catheter body.

18. An ablation and lesion feedback system, comprising:
an ablation catheter, comprising:
a catheter body having a distal end;
a hollow tip attached to the distal end of the catheter body, the hollow tip comprising an acoustically transparent shell and an electrically-conductive coating on an exterior surface of the shell;
a plurality of ribs extending inwardly from an inner surface of the shell; and
an ultrasound transducer assembly positioned within the hollow tip and mounted to rotate about a longitudinal axis of the catheter body; and
a control unit, wherein the control unit is configured to:
energize the electrically-conductive coating on the exterior surface of the shell to deliver ablating energy to a tissue to be ablated; and
operate the ultrasound transducer assembly to monitor the tissue to be ablated as the ultrasound transducer assembly rotates about a longitudinal axis of the catheter body.

19. The system according to claim 18, further comprising a radiofrequency energy source coupled to the electrically-conductive coating and the control unit.

20. The system according to claim 18, further comprising:
a transducer pinger coupled to the ultrasound transducer assembly and the control unit; and
an acoustic receiver coupled to the ultrasound transducer assembly and the control unit.
